(19) United States
(12) Patent Application Publication
(10) Pub. No.: US 2020/0368050 A1
(43) Pub. Date: Nov. 26, 2020

(54) IMPLANTABLE DEVICE WITH ENHANCED DRUG DELIVERY AREA

(71) Applicant: Envision Scientific Private Limited, Surat,Gujarat (IN)

(72) Inventors: Manish Indulal Doshi, Surat (IN); Prakash Nanjibhai Sojitra, Surat (IN); Parth Manish Doshi, Surat (IN); Dinesh Shah, Troy, MI (US)

(21) Appl. No.: 16/636,891

(22) PCT Filed: Aug. 4, 2018
(86) PCT No.: PCT/IN2018/050510

§ 371 Date: Feb. 5, 2020

(30) Foreign Application Priority Data
Aug. 5, 2017 (IN) 201721012262

(51) Int. Cl. A61F 2/90; A61F 2/95; A61F 2/958; A61L 31/10; A61L 31/16; A61L 31/12; B05D 1/02

(52) U.S. Cl. CPC A61F 2/90; A61F 2/9522; A61F 2/958; A61L 31/10; A61L 31/16; A61L 2420/02; B05D 1/02; A61F 2002/9583; A61F 2250/0067; A61F 2240/001; A61L 2300/416; A61L 31/125

(57) ABSTRACT

Disclosed is an implantable device with enhanced drug delivery area, wherein a pre-crimped stent assembly mounted on a balloon further comprises a homogenous coating of drug and associated polymeric matrix resulting in the formation of a circumferential cylindrical film formation, upon expansion of the balloon. The cylindrical film formation by the drug delivery medical devices enables maximum coverage area of the vascular lumen area, thereby preventing any untreated area within a lumen.

### FIELD OF INVENTION

The invention generally relates to a drug delivery medical apparatus. More specifically, the invention relates to a homogenously coated implantable device with enhanced drug delivery area providing maximum coverage area of target lesions within a vascular lumen.

### BACKGROUND OF INVENTION

Immense progress has been witnessed in the treatment of coronary artery disease (CAD) as a steady shift was seen from bare metal stents to drug eluting stents, the drug eluting stents (DESs) reducing restenosis at a substantially higher rate than bare metal stents. These drug eluting stents primarily used to re-open clogged arteries to re-establish blood flow and minimize rate of reoccurrence after DES implantation, have a few limitations over and above the advantages they present, with ample scope for improvement. The limitations associated with DESs specially occur in certain indications like diabetic patients, acute myocardial infarction patients, bifurcation lesion and chronic total occlusion (CTO) to name a few. For instance, the diabetic foot (below the knee patient) associated with diabetic patients experience the limitations associated with DESs.

The currently available DESs are coated on the metal surface of a stent due to which only 12-20% of the artery lumen gets delivered with a drug representative of the contact area of stent to the lumen, when implanted in the vessel wall, thereby leaving the remaining area of the lumen untreated and deficient of a drug. Furthermore, drugs with poor bioavailability and poor lipophilicity intensify the diffusion limitation in a vessel wall. As a result of the untreated area and diffusion limitations, re-blockage often occurs in the patient, the rate of blockage and re-blockage varying from patient to patient in line with the patient's body conditions and physiology. For instance, diabetic patients have higher rate of blockages than non-diabetic patients, the diabetic patients having a diffused proliferative and continuous disease type with constricted lumen diameter and lumen length further complicating drug delivery of drugs, an issue yet to be resolved.

Furthermore, from the point of view of the bioavailability of the drugs being delivered, considering the drug is coated only on the stent, minimal drug is delivered as the drug delivered is only as much as the stent is able to cover. The high bio-availability and enlarged diffusion of the drug is often compromised in this cause. For instance, although limus based drugs delivered by the existing DESs are proven to be safe, these drugs have a poor shelf-life compared to other drugs and therefore intensify focal restenosis.

Therefore, there is a need in the art for an improved and enhanced area based drug delivering device efficient in delivering drug to the entire area of a vascular lumen/artery, thereby preventing restenosis and uncontrolled cell growth from occurring.

### BRIEF DESCRIPTION OF DRAWINGS

FIG. 1 is representative of homogenous drug and polymeric matrix coating on the stent and balloon in a typical coating configuration.

FIG. 2a is representative of a cross-section of the resultant coating formation with an expanded balloon and FIG. 2b is representative of a cross-section of the resultant coating formation after the implantable device with enhanced drug delivering area is employed in a coronary vasculature.

FIG. 3 is representative of a graph based on HPLC analysis results of the sample.

FIG. 4 is representative of graph based on HPLC analysis of the control/standard solution with respect to the sample.

### DETAILED DESCRIPTION

Before describing in detail embodiments that are in accordance with the invention, it should be observed that the embodiments reside primarily in combinations of components of an improved drug delivery implantable device with homogenous drug coating on the medical apparatus. Accordingly, the components have been described to include only those specific details that are pertinent to understanding the embodiments of the invention so as not to obscure the disclosure with details that will be readily apparent to those of ordinary skill in the art having the benefit of the description herein.

In this document, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Further, before describing in detail embodiments that are in accordance with the invention, it should be observed that all the scientific and technical terms used in for describing the invention have same meanings as would be understood by a person skilled in the art.

Various embodiments of the invention provide an improved implantable drug delivery device enabling drug delivery to the entire vascular lumen area, thereby treating lesions within the lumen area in entirety and enhancing the area of drug delivery. More specifically, the improved implantable drug delivery device is a coated pre-crimped stent assembly mounted on a balloon assembly, wherein coating of the pre-crimped stent mounted on a balloon includes a homogenous cylindrical coating of a matrix of one or more drugs and one or more polymers covering a circumferential area of a vascular body lumen, on inflation/expansion of balloon assembly of the pre-crimped stent mounted on the balloon.

In accordance with present invention, the invention provides an enhanced drug delivery area implantable device delivering drug to a treatment site in the coronary and peripheral vascular artery. The coated pre-crimped stent mounted on the balloon constitutes a single consolidated drug delivery medical apparatus. The stent assembly mounted on the balloon assembly includes a plurality of strut components with a plurality of interconnected space regions defined within the plurality of the strut components, thereby creating a mesh like configuration. The crimping of a stent assembly mounted on the balloon is performed by known methods including mechanisms using stent crimping equipment and manual crimping methodologies.

Accordingly, the coating of a pre-crimped stent mounted on a balloon includes a method of coating an already pre-crimped stent mounted on a balloon. The coating covers an outer surface including an abluminal surface of the plurality of strut components of the stent assembly and regions of the balloon assembly radially extending and exposed through the plurality of interconnected space regions defined within the plurality of strut components coating an outer surface of the pre-crimped stent mounted on a balloon, wherein a plurality of sections of the balloon assembly are exposed to the coating.

The coating on the pre-crimped stent mounted on a balloon comprises an organic solvent soluble matrix of one or more drugs and one or more polymers.

The one or more drugs are selected from a group, including, but not limited to, anti-restenotic agent, an anti-proliferative agent, an anti-inflammatory agent, an antithrombotic agent, and an antioxidant an immunosuppressive agent, a cytostatic agent and a cytotoxic agent. More specifically, the one or more drugs are selected from a group, including, but not limited to, sirolimus, tacrolimus, paclitaxel, beta-estadiol, rapamycin, everolimus, ethylrapamycin, zotarolimus, ABT-578, Biolimus A9 and analogs of rapamycin mitomycin, myomycine, novolimus, permirolast potassium, alpha-interferon, bioactive RGD and salts, esters or analogues thereof.

In another exemplary embodiment, the drug may include, but is not limited to, one or more of sirolimus, tacrolimus, paclitaxel, heparin, beta-estadiol, rapamycin, everolimus, ethylrapamycin, zotarolimus, ABT-578, Biolimus A9, docetaxel and mitomycin.

The one or more polymers are selected from a group, including, but not limited to, a homopolymer; a co-polymer of glycolide and lactide; a co-polymer of trimethylene carbonate; e-caprolactone and polydiaxanone; Poly Glycolic Acid (PGA); Poly(Lactic-co-Glycolic Acid) (PLGA); Poly(Ethylene Glycol) (PEG); Polyglactin; Polyglyconate; Polydiaxanone; Polyglecaprone; Polyglycolide; Polylactide; Polyhydroxybutyrate; Poly(Glycolide-E-Caprolactone); Poly(Glycolide Trimethylene Carbonate); Poly(L-lactic Acide-L-lysine) copolymer; Tyrosine-based polyarylates; Polyiminocarbonates; Polycarbonates; Poly(D;L-lactide-Urethane); Poly(esteramide); Poly-P-Dioxanone; hyaluronic acid; chitin; chitosan; Poly-L-Glutamic Acid; Poly-L-Lysine; Polyphosphazene; Poly[bis(carboxylatophenoxy)phosphazene] and combinations thereof. In a preferred embodiment, a bio-degradable polymer matrix of Poly-L Lactide family is employed along with one or more drugs for coating the pre-crimped stent mounted on a balloon.

The method of coating the pre-crimped stent mounted on a balloon includes spray-coating a coating solution on a pre-crimped stent mounted on the balloon, the implantable device installed in a coating machine. The coating machine includes, but is not limited to, a spray nozzle unit, a protection tube, a mandrel fixture and a holder. The spray nozzle unit is used for spraying the coating solution including one or more drugs and one or more biodegradable polymer matrix dissolved in a low boiling point solvent. Further, a pre-fixed coating solution is poured in a feeding cup associated with the spray nozzle unit.

Considering a coating example, a pre-crimped stent mounted on a balloon, measuring 2.25*20 mm is installed in a coating machine. A coating solution of one or more drugs and one or more polymers is prepared and 1 ml of the coating solution is sprayed on the pre-crimped stent system mounted on the coating machine, at specific conditions including a 0.5-4.0 psi inert gas pressure, the coating machine rotating at a speed of 5-40 rpm. On spray coating the coating solution, the coating is left to dry at room temperature for a time duration of five minutes, thereby enabling the residual solvent in the coating solution to evaporate.

The coating machine may have a rotatable mandrel. The drug-delivering insertable medical device may be mounted on the rotatable mandrel and rotated along with the rotatable mandrel. The outer surface of the drug-delivering insertable medical device may be exposed to the spray nozzle unit, thereby coating the abluminal surface of the plurality of strut components of the stent assembly and regions of the balloon assembly radially extending and exposed through the plurality of interconnected space regions defined within the plurality of strut components coating an outer surface of the pre-crimped stent mounted on a balloon.

On positioning the insertable coated pre-crimped stent mounted on a balloon within a body lumen, the balloon within the pre-crimped stent mounted on the balloon is expanded at a nominal pressure range between 6 to 9 atmospheric pressure. Upon expansion of the balloon and therefore the stent, the coating on the outer surface of the pre-crimped stent mounted on the balloon expands and a homogenous cylindrical film formation of the coating for addressing target lesions within the body lumen, occurs. Considering an example of a stent measuring 2.25*20 mm includes a drug concentration ranging from 0.7 microgram per square millimeter to 1.8 microgram per square millimeter on the coating, to be delivered at a target site within a body lumen, thereby indicating enhanced drug delivery area.

The pre-crimped stent assembly on the balloon further comprises a homogenous drug and associated polymeric coating, the homogenous drug coating applied on the external surfaces of the stent and the balloon, thereby providing complete coverage of the exterior of the drug delivery medical apparatus. On inflation of the balloon within the coated pre-crimped stent mounted on the balloon, the coating is found on the plurality of strut components as well as the plurality of interconnected space regions defined within the plurality of strut components.

In a preferred embodiment, coating on a pre-crimped stent mounted on a balloon includes a sirolimus drug eluted from a polymer matrix selected from a bio-degradable matrix of a Poly-L Lactide family of copolymers. The stent assembly within the pre-crimped stent mounted on the balloon is a cobalt chromium stent with a coating on the abluminal surface of the stent and the balloon in a pre-crimped state. Coating on the abluminal surface of the stent and the balloon includes a coating on the abluminal and side surfaces of the plurality of strut components as well as the radially extending sections of balloon surface exposed through the plurality of interconnected space regions defined within the plurality of strut components of the stent assembly. Once a drug delivery device is employed within a body lumen, the biodegradable matrix of poly-L lactide degrades by hydrolysis to naturally occurring lactic acid, the naturally occurring lactic acid eventually metabolized in the body lumen to Carbon dioxide and water within a period of six to 8 months.

The coating on the pre-crimped stent including a sirolimus drug eluted from a bio-degradable matrix of a Poly-L Lactide family of copolymers is prepared from a coating solution. In an embodiment, sirolimus is dissolved in 50 ml of methanol and after complete dissolution of sirolimus in methanol, a polymer selected from a bio-degradable matrix of a Poly-L Lactide family of copolymers is also added to the solution containing sirolimus and methanol. In a next step, the solution comprising sirolimus and a selected polymer is degassed by employing an ultrasonic cleaner.

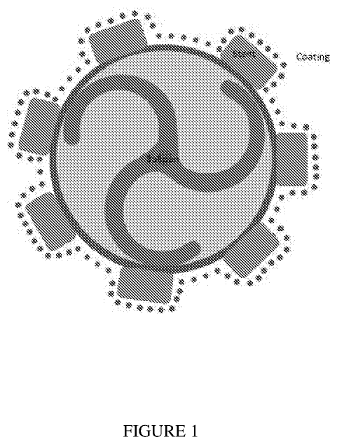

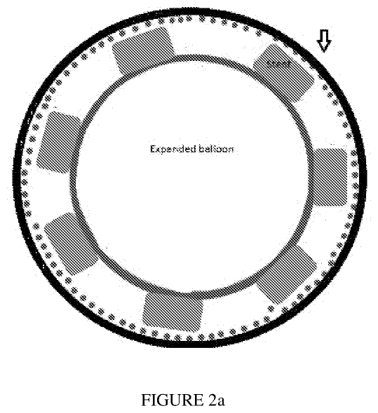

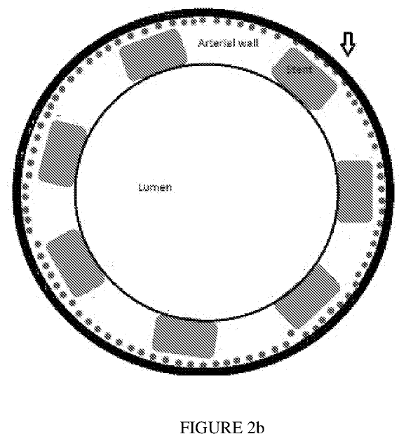

Referring to FIG. 1, a coated pre-crimped stent mounted on a balloon is illustrated in a typical coating configuration. FIG. 2a is representative of a cross-section of the resultant coating formation with an expanded/inflated balloon and FIG. 2b is representative of a cross-section of the resultant coating formation after the implantable device is employed in a coronary vasculature.

In accordance with an exemplary embodiment of the present invention, the homogenous coating of one or more drugs and an associated biodegradable polymeric matrix covers the exterior surface of the stent pre-crimped on the balloon wherein the drug delivery implantable device is deployed in a coronary artery/vasculature. In a typical procedure of deploying a drug delivery device at a target site, wherein expansion of the balloon occurs at a range from 45 seconds to 60 seconds, a homogenous cylindrical film formation of the drug and polymeric matrix coating occurs in a wet condition of the blood vessel. A typical inflation period ranging from 45 seconds to 60 seconds is maintained during deployment of stent in coronary or peripheral vascular application. As depicted in FIG. 2a, the black circle in contact with wall of the arterial wall is representative of the homogenous cylindrical film formation upon expansion of the coated balloon, thereby delivering drug to the entire lesion to have maximum coverage area.

Various embodiments include a method for addressing a plurality of lesions within a body lumen, the plurality of lesions associated with a plurality of medical conditions. The medical condition may be one or more of, restenosis, blocked body lumen, atherosclerosis, myocardial infarction and plaque accumulation in the body lumen. The body lumen may be, for example, a blood vessel, a urethra, an esophagus, a ureter and a bile duct. In a preferred embodiment, the coated pre-crimped stent mounted on a balloon addresses the lesions in a coronary/periphery artery of a diabetic patient.

In an embodiment, the coating on the outer surface of the pre-crimped stent mounted on a balloon ensures the lack of coating on the inner surface of the stent assembly and therefore the luminal surface of the plurality of strut components lack any coating. The sections of the balloon under the luminal surface of the plurality of strut components also lack coating, thereby accelerating the re-reendothelialization process, especially in a coronary or peripheral artery.

The homogenous cylindrical film formation in contact with the lumen is advantageously retained in the lumen of the coronary or peripheral artery, thereby providing a circumferential configuration. This circumferential configuration facilitates a burst drug release as well as sustained release of an appropriate drug from days to months, thereby supporting diabetic patients with the medical condition of restenosis, or reoccurrence of stenosis and further preventing uncontrolled growth of cells in lumen.

In an example, a coated pre-crimped stent mounted on a balloon includes a pre-crimped stent of size 3.00×20 mm. The amount/weight of sirolimus employed in the coating solution is 5 mg, weighed as per a precise weighing balance. The coating solution includes 5 mg of sirolimus dissolved in amber colored 100 ml Standard Measuring Flask with an addition of 25 ml methanol. The resultant coating solution is sonicated in a ultrasonic cleaner for a time duration of 2 minutes, the sonication followed by degassing of the coating solution. The theoretical standard solution concentration of the coating solution after degassing of the coating solution is 50 μg/ml and a control/standard is also prepared for comparative analysis.

Accordingly, a pre-crimped stent mounted on a balloon is dipped in a 10 ml amber colored standard measuring flask containing the coating solution. The coating solution is filtered with 0.45 membrane filter using syringe filter and filled in an HPLC vial for further analysis. The HPLC system for analysis includes a UV-VIS Detector and a Column: BDS HYPERSIL C18, wherein the dimensions include 250×4.6 mm and a particle Size of 5 μm was used. The operating parameters further include a flow rate at 1 ml/min, λ maxima at 277 nm, an auto sampler injection volume of 20 μl, column temperature at 40° C. (±2° C.), sample temperature at 15° C. (±1° C.) and a run time of 12 minutes.

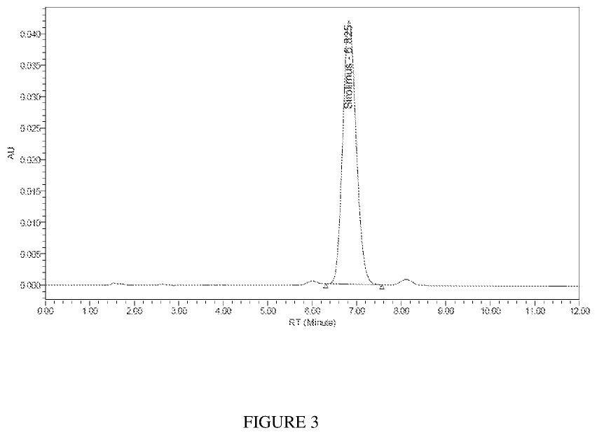

FIG. 3 is illustrative of a graph based on HPLC analysis results of the sample. Referring to FIG. 3, the average area coverage by the drug after injection of the coating solution is 878217.

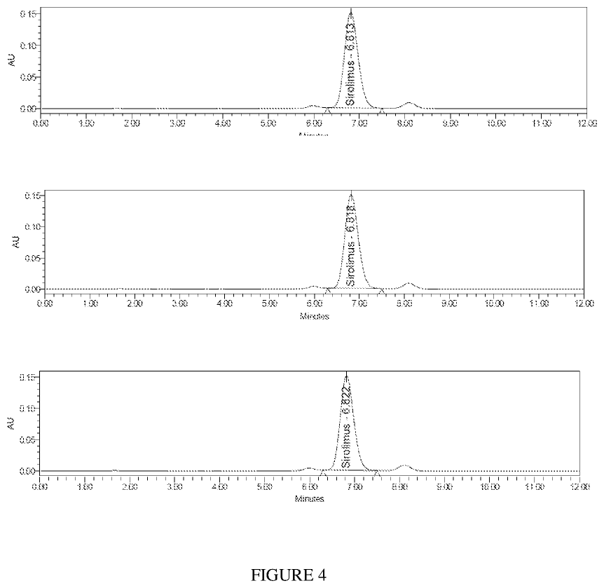

FIG. 4 is illustrative of a graph based on HPLC analysis of the control/standard solution. Referring to FIG. 4, the average area coverage with respect to the control/standard solution is 3147981.

The amount of drug coated on the pre-crimped stent mounted on the balloon for the sample with respect to the standard solution/control is calculated by using the formula:

$$Drug content = \frac{sample area}{standard area} \times \frac{standard weight}{dilution} \times \frac{Dilution}{sample} \times Potency .$$

Accordingly, the drug content calculated using the average area coverage for the sample and average area coverage for the standard solution/control is 136.56 μg pre-crimped stent measuring 3.00×20 mm.

In another embodiment, the homogenously pre-coated implantable device with enhanced drug delivery area of a pre-crimped stent assembly mounted on a balloon is employed in the treatment of acute myocardial infraction (AMI) patients and thrombus containing lesion (TCL) patients. The existing methodology of treating AMI patients and TCL patients include thrombolysis by providing streptokinase to dissolve the thrombus. In another method a thrombus aspiration catheter is used to aspirate thrombus from the lesion followed by implantation of a DES. Although these methodologies are successful in removing the thrombus after implantation, a no-flow or slow-flow situation is often created due to stent thrombus or debris inside the lesion. Considering the high tendency of reoccurrence of acute thrombus formation on implantation of DES exists, there requires a need to evade sub-acute or late thrombus after implantation of DES. Therefore, on employing the drug delivery implantable device in accordance with the present invention, the moderate tensile strength of the homogenous cylindrical film formation resists the occurrence of acute, sub-acute as well as late thrombus formation in the lumen, further eliminating the slow-flow and no-flow conditions.

Various embodiments of the present invention advantageously provide a circumferential moderate tensile strength film formation of a drug and associated polymeric matrix coating providing maximum coverage of lesions within a lumen in the absence of restenosis and prevention of acute, sub-acute and late thrombus formation within the vascular lumen area. The present invention therefore combines the benefits of a drug eluting stent as well as the benefits of a drug eluting balloon and increases the availability of a drug within a body lumen.

Those skilled in the art will realize that the above-recognized advantages and other advantages described herein are merely exemplary and are not meant to be a complete rendering of all of the advantages of the various embodiments of the invention.

In the foregoing provisional specification, specific embodiments of the invention have been described. However, one of ordinary skill in the art appreciates that various modifications and changes can be made to the invention without deviating from the scope of the invention. Accordingly, the provisional specification is to be regarded in an illustrative rather than a restrictive sense, and all such modifications are intended to be included within the scope of the invention.

1-11. (canceled)

12. A method of coating a stent comprising:
crimping a stent assembly on a balloon assembly to form a pre-crimped stent mounted on a balloon, the stent assembly comprising a plurality of strut components with a plurality of interconnected space regions defined within the plurality of strut components; and
covering an outer surface of the pre-crimped stent mounted on the balloon with a coating, wherein a plurality of sections of the balloon assembly are exposed to the coating, forming a homogenous cylindrical coating.

13. The method of claim 12, wherein covering the pre-crimped stent mounted on the balloon further comprises applying the coating by spray coating.

14. The method of claim 13, wherein the spray coating further comprises rotational spray coating the pre-crimped stent mounted on the balloon at a speed of between 5 and 40 rotations per minute.

15. The method of claim 14, wherein the spray coating further comprises spray coating in an environment comprising between 0.5 and 4.0 psi inert gas pressure.

16. The method of claim 13, wherein the coating comprises an organic solvent soluble matrix of one or more drugs and one or more polymers.

17. The method of claim 16, wherein the coating includes a solution comprising a matrix of one or more drugs and one or more polymers.

18. The method of claim 17, wherein the coating includes the one or more drugs and the one or more polymers dissolved in fast evaporating solvents.

19. The method of claim 12, wherein covering the outer surface of the pre-crimped stent mounted on the balloon enables an inner surface of the pre-crimped stent mounted on the balloon to be devoid of coating.

20. The method of claim 12, further comprising expanding the balloon within a body lumen at a pressure in a range of between 6 and 9 atmospheric pressure.

21. The method of claim 20, further comprising applying the coating to surfaces of the body lumen.

22. An apparatus comprising:
a pre-crimped stent mounted on a balloon; and
a coating surrounding the pre-crimped stent mounted on the balloon, wherein the coating comprises a homogenous cylindrical coating of a matrix of one or more drugs and one or more polymers, and wherein the coating covers a circumferential area of a vascular body lumen on expansion of the balloon.

23. The apparatus of claim 22, wherein the homogenous cylindrical coating addresses lesions within the vascular body lumen.

24. The apparatus of claim 22, wherein the one or more drugs comprises at least one drug selected from a group consisting of an anti-restenotic agent, an anti-proliferative agent, an anti-inflammatory agent, an antithrombotic agent, an antioxidant, an immunosuppressive agent, a cytostatic agent, and a cytotoxic agent.

25. The apparatus of claim 22, wherein the one or more drugs comprises at least one drug selected from a group consisting of sirolimus, tacrolimus, paclitaxel, beta-estadiol, rapamycin, everolimus, ethylrapamycin, zotarolimus, ABT-578, Biolimus A9 and analogs of rapamycin mitomycin, myomycine, novolimus, permirolast potassium, alpha-interferon, bioactive RGD and salts, esters or analogues thereof.

26. The apparatus of claim 22, wherein the one or more polymers comprises at least one polymer selected from the group consisting of a homopolymer, a co-polymer of glycolide and lactide, a co-polymer of trimethylene carbonate, e-caprolactone and polydiaxanone, Poly Glycolic Acid (PGA), Poly(Lactic-co-Glycolic Acid) (PLGA), Poly(Ethylene Glycol) (PEG), Polyglactin, Polyglyconate, Polydiaxanone, Polyglecaprone, Polyglycolide, Polylactide, Polyhydroxybutyrate, Poly(Glycolide-E-Caprolactone), Poly(Glycolide Trimethylene Carbonate), Poly(L-lactic Acide-L-lysine) copolymer, Tyrosine-based polyarylates, Polyiminocarbonates, Polycarbonates, Poly(D;L-lactide-Urethane), Poly(esteramide), Poly-P-Dioxanone, hyaluronic acid, chitin, chitosan, Poly-L-Glutamic Acid, Poly-L-Lysine, Polyphosphazene, and Poly[bis(carboxylatophenoxy)phosphazene], and combinations thereof.

27. A system comprising:
a balloon insertable into a blood vessel, wherein the balloon is configured to expand and contact inner walls of the blood vessel;
a stent coupled to the balloon; and
a coating surrounding the stent and the balloon, wherein the coating comprises one or more drugs and one or more polymers.

28. The system of claim 27, wherein the coating addresses lesions within the blood vessel.

29. The system of claim 27, wherein the one or more drugs comprises at least one drug selected from a group consisting of an anti-restenotic agent, an anti-proliferative agent, an anti-inflammatory agent, an antithrombotic agent, an antioxidant, an immunosuppressive agent, a cytostatic agent, and a cytotoxic agent.

30. The system of claim 27, wherein the one or more drugs comprises at least one drug selected from a group consisting of sirolimus, tacrolimus, paclitaxel, beta-estadiol, rapamycin, everolimus, ethylrapamycin, zotarolimus, ABT-578, Biolimus A9 and analogs of rapamycin mitomycin, myomycine, novolimus, permirolast potassium, alpha-interferon, bioactive RGD and salts, esters or analogues thereof.

31. The system of claim 27, wherein the one or more polymers comprises at least one polymer selected from the group consisting of a homopolymer, a co-polymer of glycolide and lactide, a co-polymer of trimethylene carbonate, e-caprolactone and polydiaxanone, Poly Glycolic Acid (PGA), Poly(Lactic-co-Glycolic Acid) (PLGA), Poly(Ethylene Glycol) (PEG), Polyglactin, Polyglyconate, Polydiaxanone, Polyglecaprone, Polyglycolide, Polylactide, Polyhydroxybutyrate, Poly(Glycolide-E-Caprolactone), Poly(Glycolide Trimethylene Carbonate), Poly(L-lactic Acide-L-lysine) copolymer, Tyrosine-based polyarylates, Polyiminocarbonates, Polycarbonates, Poly(D;L-lactide-Urethane), Poly(esteramide), Poly-P-Dioxanone, hyaluronic acid, chitin, chitosan, Poly-L-Glutamic Acid, Poly-L-Lysine, Polyphosphazene, and Poly[bis(carboxylatophenoxy)phosphazene], and combinations thereof.
